(19) United States
(12) Patent Application Publication
(10) Pub. No.: US 2009/0105481 A1
(43) Pub. Date: Apr. 23, 2009

(54) Asymmetric Catalytic Hydrogenation of Prochiral Ketones and Aldehydes

(76) Inventors: Felix Spindler, Starrkirch-Wil (CH); Ulrike Nettekoven, Allschwil (CH); Mauro Perseghini, Pambio-Noranco (CH)

Correspondence Address: WENDEROTH, LIND & PONACK, L.L.P., 1030 15th Street, N.W.,, Suite 400 East, Washington, DC, 20005-1503, US

(21) Appl. No.: 12/224,826

(22) PCT Filed: Mar. 8, 2007
(86) PCT No.: PCT/EP2007/052160

§ 371 Date: Sep. 8, 2008

(51) Int. Cl. C07C 37/00; C07F 9/653; C07C 35/08

(52) U.S. Cl. 548/119; 568/799; 556/19; 568/832

(57) ABSTRACT

Process for stereoselective hydrogenation by reacting racemic aldehydes or ketones having a stereogenic carbon atom in the position relative to the C(O) group and containing the structural element —(O)C—C—CH— by means of hydrogen in the presence of a base and a ruthenium complex containing a bidentate ligand having coordinating P and N atoms, a monophosphine ligand and anionic and/or uncharged ligands as homogeneous catalyst, with the charge being balanced by one or two monovalent acid anions or a divalent acid anion when uncharged ligands are present.

The present invention relates to a process for the enantioselective or diastereoselective, homogeneous hydrogenation of asymmetric aldehydes or ketones having a stereogenic α carbon atom to the keto group to alcohols using ruthenium complexes which contain a bidentate ligand having P and N atoms and a monophosphine ligand, in the presence of hydrogen and a base. The invention also relates to 1-sec-phosphino-2-oxazolidinyl-ferrocenes having P-bonded, ortho-substituted aryl groups.

WO 2004/050585 describes the catalytic hydrogenation of ketones or ketimines by means of hydrogen in the presence of a base and a pentacoordinated ruthenium complex as catalyst or catalyst precursor containing a monophosphine and a bidentate P̂N ligand as ligand. The hydrogenation leads to high chemical conversions at high catalyst activities and, when prochiral ketones are used, to very good stereoselectivities or high optical yields.

It has now surprisingly been found that racemates of aldehydes or ketones having a stereogenic carbon atom in the α position relative to the keto group can be converted in the asymmetric hydrogenation according to WO 2004/050585 via simultaneous dynamic-kinetic or kinetic racemate resolution into predominantly one enantiomeric primary alcohol or into predominantly one diastereomeric carbinol. Unexpectedly, only one enantiomer is hydrogenated in the hydrogenation of these aldehydes and ketones and the presence of a base results in continual racemization of the other enantiomer to establish equilibrium rapidly, so that high diastereomer ratios and enantiomeric excesses can surprisingly be achieved. The ruthenium catalyst can here contain either achiral or chiral ligands.

The invention firstly provides a process for preparing a predominantly enantiomeric primary alcohol or a predominantly diastereomeric secondary alcohol by reacting aldehydes or ketones with hydrogen in the presence of a base and a ruthenium complex containing a bidentate ligand having coordinating P and N atoms, a monophosphine ligand and anionic and/or uncharged ligands as homogeneous catalyst, with the charge being balanced by one or two monovalent acid anions or a divalent acid anion when uncharged ligands are present, which is characterized in that a racemic aldehyde or ketone which has a stereogenic carbon atom in the α position relative to the C(O) group and has the structural element —(O)C—C*—CH is reacted.

In the structural element —(O)C—C*—CH, C* is the stereogenic carbon atom in the α position.

The ruthenium complexes can be prepared either “in situ” prior to the hydrogenation in a separate solution or in the reaction solution before addition of or in the presence of a substrate by addition of ligands to ruthenium complexes or salts, or they can be prepared and isolated as complexes beforehand and then used as isolated compound. The ruthenium complexes can, for example, be prepared by methods described by S. Uemura et al. in Organometallics 1999, 18, 2291.

The hydrogenation process of the invention can be carried out at customary pressures, for example from 1·10⁵ to 1·10⁷ Pa (from 1 to 100 bar). It is advantageous to use a pressure of from 2·10⁶ to 8.5·10⁶ Pa (from 20 to 85 bar), in particular from 4·10⁶ to 8·10⁶ Pa (from 40 to 80 bar).

The choice of reaction temperature is dependent essentially on the solubility of the reactants and ruthenium complexes in the solvents used. The reaction temperature can be, for example, from 0° C. to 100° C. At higher temperatures, undesirable racemization can occur and the reaction temperature is therefore advantageously selected in the range from 10 to 60° C. The hydrogenation is particularly preferably carried out at about room temperature, very particularly preferably at a temperature of from 20 to 35° C.

The process of the invention can be carried out without solvent or in the presence of an inert solvent. Suitable solvents are, for example, aliphatic, cycloaliphatic and aromatic hydrocarbons (pentane, hexane, petroleum ether, cyclohexane, methylcyclohexane, benzene, toluene, xylene, mesitylene), aliphatic halogen hydrocarbons (methylene chloride, chloroform, dichloroethane and tetrachloroethane), nitriles (acetonitrile, propionitrile, benzonitrile), ethers (diethyl ether, dibutyl ether, t-butyl methyl ether, ethylene glycol dimethyl ether, ethylene glycol diethyl ether, diethylene glycol dimethyl ether, tetrahydrofuran or dioxane), carboxylic esters and lactones (ethyl or methyl acetate, valerolactone), N-substituted lactams (N-methylpyrrolidone), carboxamides (dimethylacetamide, dimethylformamide), acyclic ureas (tetramethylurea) or cyclic ureas (dimethylimidazolidinone), sulphoxides and sulphones (dimethyl sulphoxide, dimethyl sulphone, tetramethylene sulphoxide, tetramethylene sulphone), alcohols (methanol, ethanol, propanol, butanol, ethylene glycol monoethyl ether, diethylene glycol monomethyl ether) and water. The solvents can be used either alone or as mixtures of at least two solvents.

As bases, it is possible to use either inorganic bases or organic nitrogen bases, for example alkaline earth metal or alkali metal hydroxides, alkali metal alkoxides, alkali metal carbonates or hydrogencarbonates, alkali metal amides or quaternary ammonium salts. Preferred bases are KOH, KOCH₃, KO-i-C₃H₇, KO-t-C₄H₉, LiOH, LiOCH₃, LiO-i-C₃H₇, NaOH, NaOCH₃, NaO-i-C₃H₇, LiNH₂ or NaNH₂, or LiN(CH₃)₂ or NaN(CH₃)₂. The bases can be used in solid form or as solutions, for example in an alcohol such as methanol, ethanol, n- or i-propanol or n-, i- or t-butanol, water or mixtures of such an alcohol and water. Furthermore, the bases can be used within a wide concentration range. The molar ratio of base to substrate can be, for example, from 10 to 0.1, more preferably from 5 to 0.5.

The ruthenium complexes are used in catalytic amounts. The molar ratio of substrate to ruthenium complex can be from 106 to 20 and preferably from 105 to 50.

Suitable ruthenium complexes are comprehensively described in WO 2004/050585. They can, for example, correspond to the general formula I,

[XYRu(PR₁R₂R₃)(P-Z-N)] (I),

where
X and Y are each, independently of one another, a hydride, halide, C₁₋₈-alkoxide or C₁₋₈-acyloxy or a coordinated organic solvent ligand containing at least one heteroatom from the group consisting of O, S and N, with a resulting cationic complex having one or two solvent ligands being neutralized by one or two monovalent anions or a divalent anion,
R₁, R₂ and R₃ are each, independently of one another, a hydrocarbon radical or a C-bonded heterohydrocarbon radical having heteroatoms selected from the group consisting of O, S, N, NH and N(C₁-C₄-alkyl), each of which has from 1 to 22, preferably from 1 to 14 and particularly preferably from 1 to 10, carbon atoms and is unsubstituted or substituted, or one of the radicals R₁, R₂, R₃ is as defined above and the remaining two radicals together with the phosphorus atom and carbon atoms form a 4- to 8-membered, unsubstituted or substituted ring, and
P-Z-N is a bidentate ligand of the formula (II),

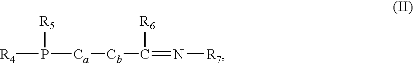

where
R₄ and R₅ are each, independently of one another, a hydrocarbon radical or a C-bonded heterohydrocarbon radical having heteroatoms selected from the group consisting of O, S, N, NH or N(C₁-C₄-alkyl), each of which has from 1 to 22, preferably from 1 to 14 and particularly preferably from 1 to 10, carbon atoms, or R₄ and R₅ together with the phosphorus atom and further carbon atoms form a 4- to 8-membered ring, with R₄ and R₅ being unsubstituted or substituted,
Cₐ and C_{b} together are part of a five- or six-membered arene or heteroarene which is unsubstituted or substituted by OH, F, Cl, Br, —CN, C₁₋₄-alkyl, C₁₋₄-alkoxy, trifluoromethyl, trifluoromethoxy, phenyl, C₁₋₄-alkyl or C₁₋₄-alkoxyphenyl, —C(O)O—C₁₋₄-alkyl or di(C₁-C₄-alkyl)amino,
R₆ is a hydrogen atom, a linear, branched or cyclic C₁₋₁₀-alkyl or C₂₋₁₀-alkenyl group or a C₆₋₁₀-aryl group, each of which is unsubstituted or substituted by C₁₋₄-alkyl, C₁₋₄-alkoxy, trifluoromethyl, trifluoromethoxy, di(C₁-C₄-alkyl)amino, phenyl, benzyl, C₁₋₄-alkylphenyl or C₁₋₄-alkylbenzyl, or R₆ is a —OR_{6′} or —NR_{6′}R_{6″} radical, where R_{6′} and R_{6″} have the same meanings as R₆,
R₇ is a hydrogen atom, a linear, branched or cyclic C₁₋₁₀-alkyl or C₂₋₁₀-alkenyl group, or R₇ is a R_{7′}CO— or R_{7′}SO₂— radical, where R_{7′} is a hydrocarbon radical having from 1 to 14 carbon atoms, or
R₆ and R₇ together with the group —C═N— form an unsaturated five- to ten-membered, preferably five- to seven-membered, substituted or unsubstituted heterocycle.

By way of explanation, it may be stated that a five-membered arene can also be a cyclopentadienyl ring in a metallocene, for example ferrocene.

In the complexes of the formula I, X and Y are each preferably halide such as chloride, bromide and iodide, with chloride being particularly preferred. Examples of alkoxy and acyloxy groups X and Y are methoxy, ethoxy, n- and i-propoxy, n-, i- and t-butoxy, formyloxy, acetyloxy, propionyloxy, butyryloxy and phenyloxy. Suitable solvent ligands have been mentioned above under solvents. Suitable anions for neutralization are, for example, SO₄²⁻, CN⁻, OCN⁻, BF₄⁻, PF₆⁻, F₃C—SO₂O⁻, HC(O)O⁻ or CH₃C(O)O⁻.

The hydrocarbon or heterohydrocarbon radicals R₁, R₂, R₃, R₄ and R₅ can be unsubstituted or be substituted by 1, 2 or 3 radicals. Preferred substituents are selected from among C₁₋₄-alkyl, C₁₋₄-alkoxy, trifluoromethyl, trifluoromethoxy and di(C₁-C₄-alkyl)amino. R₁, R₂, R₃ and also R₄ and R₅ are preferably identical radicals.

Preferred radicals R₁, R₂, R₃, R₄ and R₅ are radicals selected from the group consisting of linear or branched C₁-C₁₂-alkyl; unsubstituted or C₁-C₆-alkyl or C₁-C₆-alkoxy-substituted C₅-C₁₂-cycloalkyl or C₅-C₁₂-cycloalkyl-CH₂—; phenyl, naphthyl, furyl or benzyl; and phenyl or benzyl substituted by halogen (for example F, Cl and Br), C₁-C₆-alkyl, C₁-C₆-haloalkyl (for example trifluoromethyl), C₁-C₆-alkoxy, C₁-C₆-haloalkoxy (for example trifluoromethoxy), (C₆H₅)₃Si, (C₁-C₁₂-alkyl)₃Si, sec-amino or —CO₂—C₁-C₆-alkyl (for example —CO₂CH₃).

Examples of alkyl radicals R₁, R₂, R₃, R₄ and R₅, which preferably contain from 1 to 6 carbon atoms, are methyl, ethyl, n-propyl, i-propyl, n-butyl, i-butyl, t-butyl and the isomers of pentyl and hexyl. Examples of unsubstituted or alkyl-substituted cycloalkyl substituents on P are cyclopentyl, cyclohexyl, methylcyclopentyl and ethylcyclopentyl, dimethylcyclopentyl, methylcyclohexyl and ethylcyclohexyl and dimethylcyclohexyl. Examples of alkyl-, alkoxy-, haloalkyl-, haloalkoxy- and halogen-substituted phenyl and benzyl substituents on P are o-, m- or p-fluorophenyl, o-, m- or p-chlorophenyl, difluorophenyl or dichlorophenyl, pentafluorophenyl, methylphenyl, dimethylphenyl, trimethylphenyl, ethylphenyl, methylbenzyl, methoxyphenyl, dimethoxyphenyl, trifluoromethylphenyl, bistrifluoromethylphenyl, tristrifluoromethylphenyl, trifluoromethoxyphenyl, bistrifluoromethoxyphenyl and 3,5-dimethyl-4-methoxyphenyl.

Preferred radicals R₁, R₂, R₃, R₄ and R₅ are selected from the group consisting of C₁-C₆-alkyl, unsubstituted cyclopentyl or cyclohexyl or cyclopentyl or cyclohexyl substituted by from 1 to 3 C₁-C₄-alkyl or C₁-C₄-alkoxy radicals, benzyl and in particular phenyl, which are unsubstituted or substituted by from 1 to 3 C₁-C₄-alkyl, C₁-C₄-alkoxy, F, Cl, C₁-C₄-fluoroalkyl or C₁-C₄-fluoroalkoxy radicals. The substitutent F can also be present another four or five times.

Preferred radicals R₁, R₂, R₃, R₄ and R₅ are selected from the group consisting of linear or branched C₁-C₆-alkyl, unsubstituted cyclopentyl or cyclohexyl or cyclopentyl or cyclohexyl substituted by from one to three C₁-C₄-alkyl or C₁-C₄-alkoxy radicals, furyl, norbornyl, adamantyl, unsubstituted benzyl or benzyl substituted by from one to three C₁-C₄-alkyl or C₁-C₄-alkoxy radicals and in particular unsubstituted phenyl or phenyl substituted by from one to three C₁-C₄-alkyl, C₁-C₄-alkoxy, —NH₂, —N(C₁-C₆-alkyl)₂, OH, F, Cl, C₁-C₄-fluoroalkyl or C₁-C₄-fluoroalkoxy radicals.

R₁, R₂, R₃, R₄ and R₅ are particularly preferably radicals selected from the group consisting of C₁-C₆-alkyl, cyclopentyl, cyclohexyl, furyl and unsubstituted phenyl or phenyl substituted by from one to three C₁-C₄-alkyl, C₁-C₄-alkoxy and/or C₁-C₄-fluoroalkyl radicals.

In the case of R₁ and R₂ or R₄ and R₅ together, the resulting group can be cyclic sec-phosphino, for example a group having one of the formulae

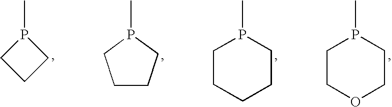

which are unsubstituted or substituted by one or more —OH, C₁-C₈-alkyl, C₄-C₈-cycloalkyl, C₁-C₆-alkoxy, C₁-C₄-alkoxy-C₁-C₄-alkyl, phenyl, C₁-C₄-alkylphenyl or C₁-C₄-alkoxyphenyl, benzyl, C₁-C₄-alkylbenzyl or C₁-C₄-alkoxybenzyl, benzyloxy, C₁-C₄-alkylbenzyloxy or C₁-C₄-alkoxybenzyloxy or C₁-C₄-alkylidenedioxyl groups.

The substituents can be bound in one or both α positions relative to the P atom in order to introduce chiral carbon atoms. The substituents in one or both α positions are preferably C₁-C₄-alkyl or benzyl, for example methyl, ethyl, n- or i-propyl, benzyl or —CH₂—O—C₁-C₄-alkyl or —CH₂—O—C₆-C₁₀-aryl.

Substituents in the β, γ positions can be, for example, C₁-C₄-alkyl, C₁-C₄-alkoxy, benzyloxy, —OH or —O—CH₂—O—, —O—CH(C₁-C₄-alkyl)-O—, —O—C(C₁-C₄-alkyl)₂-O— and —O—CH—(C₆-C₁₀-aryl)-O—. Some examples are methyl, ethyl, methoxy, ethoxy, —O—CH(phenyl)-O—, —O—CH(methyl)-O— and —O—C(methyl)₂-O—.

An aliphatic 5- or 6-membered ring or benzene can be fused onto two adjacent carbon atoms in the radicals of the above formulae.

Other known and suitable secondary phosphino radicals are those derived from cyclic and chiral phospholanes having seven carbon atoms in the ring, for example those of the formulae

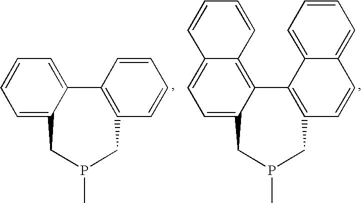

in which the aromatic rings may be substituted by C₁-C₄-alkyl, C₁-C₄-alkoxy, C₁-C₄-alkoxy-C₁-C₂-alkyl, phenyl, benzyl, benzyloxy or C₁-C₄-alkylidenedioxyl or C₁-C₄-alkylenedioxyl (see US 2003/0073868 A1 and WO 02/048161).

Depending on the type of substitution and number of substituents, the cyclic phosphino radicals can be C-chiral, P-chiral or C- and P-chiral.

The cyclic sec-phosphino radical can, for example, correspond to one of the formulae (only one of the possible diastereomers shown),

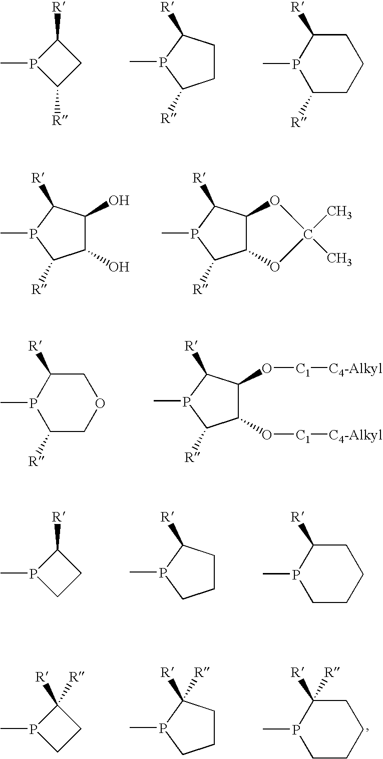

where
the radicals R′ and R″ are each C₁-C₄-alkyl, for example methyl, ethyl, n- or i-propyl, benzyl or —CH₂—O—C₁-C₄-alkyl or —CH₂—O—C₆-C₁₀-aryl, and R′ and R″ can be identical or different. If R′ and R″ are bound to the same carbon atom, they can also together be C₄-C₅-alkylene.

In a preferred embodiment, R₁, R₂, R₃, R₄ and R₅ are preferably acyclic sec-phosphino selected from the group consisting of —P(C₁-C₆-alkyl)₂, —P(C₅-C₈-cycloalkyl)₂, —P(C₇-C₁₂-bicycloalkyl)₂, —P(o-furyl)₂, —P(C₆H₅)₂, —P[2-(C₁-C₆-alkyl)C₆H₄]₂, —P[3-(C₁-C₆-alkyl)C₆H₄]₂, —P[4-(C₁-C₆-alkyl)C₆H₄]₂, —P[2-(C₁-C₆-alkoxy)C₆H₄]₂, —P[3-(C₁-C₆-alkoxy)C₆H₄]₂, —P[4-(C₁-C₆-alkoxy)C₆H₄]₂, —P[2-(trifluoromethyl)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(C₁-C₆-alkyl)₂C₆H₃]₂, —P[3,5-bis(C₁-C₆-alkoxy)₂C₆H₃]₂, —P[3,4,5-tris(C₁-C₆-alkoxy)₂C₆H₂]₂ and —P[3,5-bis(C₁-C₆-alkyl)₂-4-(C₁-C₆-alkoxy)C₆H₂]₂, or cyclic phosphine selected from the group consisting of

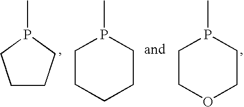

which are unsubstituted or substituted by one or more C₁-C₄-alkyl, C₁-C₄-alkoxy, C₁-C₄-alkoxy-C₁-C₂-alkyl, phenyl, benzyl, benzyloxy, hydroxy, C₁-C₄-alkylidenedioxyl or unsubstituted or phenyl-substituted methylenedioxyl groups.

Some specific examples are —P(CH₃)₂, —P(i-C₃H₇)₂, —P(n-C₄H₉)₂, —P(i-C₄H₉)₂, —P(C₆H₁₁)₂, —P(norbornyl)₂, —P(o-furyl)₂, —P(C₆H₅)₂, P[2-(methyl)C₆H₄]₂, P[3-(methyl)C₆H₄]₂, —P[4-(methyl)C₆H₄]₂, —P[2-(methoxy)C₆H₄]₂, —P[3-(methoxy)C₆H₄]₂, —P[4-(methoxy)C₆H₄]₂, —P[3-(trifluoromethyl)C₆H₄]₂, —P[4-(trifluoromethyl)C₆H₄]₂, —P[3,5-bis(trifluoromethyl)C₆H₃]₂, —P[3,5-bis(methyl)C₆H₃]₂, —P[3,5-bis(methoxy)C₆H₃]₂, —P[3,4,5-tri(methoxy)C₆H₂]₂, —P[3,5-bis(methyl)₂₋₄-(methoxy)C₆H₂]₂ and groups of the formulae

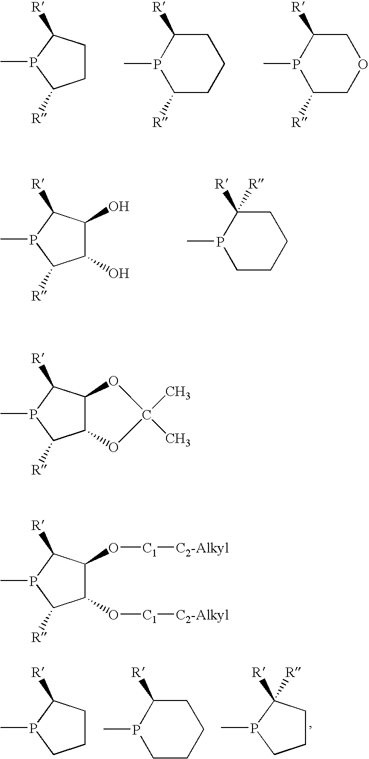

where
R′ is methyl, ethyl, methoxy, ethoxy, phenoxy, benzyloxy, methoxymethyl, ethoxymethyl or benzyloxymethyl and R″ independently has one of the meanings of R′.

In a preferred embodiment of the process of the invention, R₄ and R₅ are each phenyl or C-bonded heteroaryl having heteroatoms selected from the group consisting of O, S, NH, N or N—C₁₋₄-alkyl, each of which is substituted in at least one ortho position relative to the P—C bond by C₁₋₆-alkyl, C₁₋₆-alkoxy, C₁₋₆-alkylthio, phenyl, phenoxy, benzyl, benzyloxy, C₁₋₆-fluoroalkyl, F, Cl, Br, OH or N(C₁₋₆-alkyl)₂ or onto which an aliphatic, heteroaliphatic, aromatic or heteroaromatic five- or six-membered ring is fused in the 2,3 positions and in the case of heteroaryl also in the 3,4 positions, with the phenyl or heteroaryl also being able to contain further substituents.

Cₐ and C_{b} together with further carbon atoms or carbon atoms and heteroatoms can be derived from five- or six-membered arene or heteroarene, for example from benzene, naphthalene, anthracene, thiophene, benzothiophene, furan, benzofuran, pyridine, N—C₁₋₄-alkylpyrrole, indole, quinoline, isoquinoline or metallocenes, in particular ferrocene. Particular preference is given to phenylene and ferrocenylene.

R₆ can be, for example, a hydrogen atom, C₁₋₈-alkyl, C₅₋₈-cycloalkyl, C₅₋₈-cycloalkyl-C₁₋₄-alkyl, C₄₋₇-heterocycloalkyl or C₄₋₇-heterocycloalkyl-C₁₋₄-alkyl having heteroatoms selected from the group consisting of O, S, NH or N—C₁₋₄-alkyl, phenyl, naphthyl, benzyl, phenylethyl, thiophenyl, benzothiophenyl, furanyl, benzofuranyl, pyridinyl, N—C₁₋₄-alkylpyrryl, indolyl, quinolinyl or isoquinolinyl, each of which is unsubstituted or as defined above.

R₇ is preferably a hydrogen atom, C₁₋₈-alkyl, C₅₋₈-cycloalkyl. R_{7′} is preferably C₁₋₈-alkyl, C₅₋₈-cycloalkyl, C₆₋₁₀-aryl or C₇₋₁₂-aralkyl, for example methyl, ethyl, n- or i-propyl, n-, i- or t-butyl, cyclohexyl, phenyl or benzyl.

R₆ and R₇ together with the group —C═N— preferably form an unsaturated five- or six-membered, substituted or unsubstituted heterocycle which particularly preferably corresponds to the formula III,

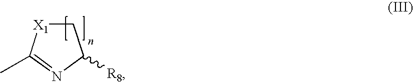

where
n is 1 or 2,
X₁ is O, S, N, NH or N—C₁₋₄-alkyl and
R₈ is C₁₋₆-alkoxy-C₁₋₄-alkyl, linear or branched C₁₋₈-alkyl, C₅₋₈-cycloalkyl, C₅₋₈-cycloalkyl-C₁₋₈-alkyl, C₆₋₁₄-aryl, C₇₋₁₂-aralkyl, C₃₋₁₂-heteroaryl, C₄₋₁₆-heteroaralkyl, where the cyclic radicals are unsubstituted or substituted by OH, F, Cl, Br, —CN, C₁₋₄-alkyl, C₁₋₄-alkoxy, trifluoromethyl, trifluoromethoxy, phenyl, C₁₋₄-alkylphenyl or C₁₋₄-alkoxyphenyl, —C(O)O—C₁₋₄-alkyl or di(C₁-C₄-alkyl)amino.

Particularly preferred radicals of the formula III are those in which n is 1 and X₁ is O and R₈ is C₁₋₄-alkoxy-C₁₋₂-alkyl, linear or branched C₁₋₆-alkyl, phenyl or benzyl. Examples of R₈ are methoxymethyl, ethoxymethyl, methoxyethyl, ethoxyethyl, n-propoxymethyl, i-propoxymethyl, n-propoxyethyl, i-propoxyethyl, n-butoxymethyl, i-butoxymethyl, t-butoxymethyl, n-butoxyethyl, i-butoxyethyl, t-butoxyethyl, methyl, ethyl, n- or i-propyl, n-, i- or t-butyl, cyclohexyl, cyclohexylmethyl, phenyl and benzyl.

In a preferred embodiment of the process of the invention, the ligands of the formula II preferably correspond to the formula IV,

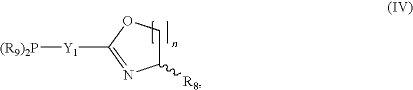

where
Y₁ is a ferrocene radical of the formula (A) which may be unsubstituted or be substituted by C₁₋₄-alkyl or halogen (for example F, Cl, Br or methyl) or a radical of the formula (B) which may be unsubstituted or be substituted by OH, F, Cl, Br, —CN, C₁₋₄-alkyl, C₁₋₄-alkoxy, trifluoromethyl, trifluoromethoxy, phenyl, C₁₋₄-alkylphenyl or C₁₋₄-alkoxyphenyl, —C(O)O—C₁₋₄-alkyl or di(C₁-C₄-alkyl)amino:

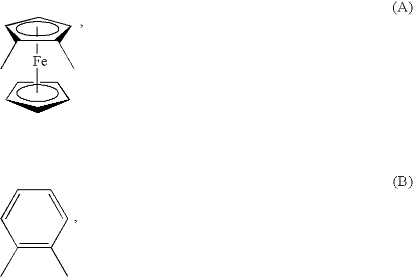

n is 1 or 2, preferably 1,
R₈ is C₁₋₄-alkoxy-C₁₋₂-alkyl, linear or branched C₁₋₄-alkyl, cyclohexyl, phenyl or benzyl, where the cyclic radicals may be substituted as defined above, and
the radicals R₉ are each phenyl or C-bonded heteroaryl having heteroatoms selected from the group consisting of O, S, N, NH or N—C₁₋₄-alkyl, each of which is either substituted in at least one ortho position relative to the P—C bond by C₁₋₆-alkyl, C₁₋₆-alkoxy, C₁₋₆-alkylthio, phenyl, phenoxy, benzyl, benzyloxy, C₁₋₆-fluoroalkyl, F, Cl, Br, OH or N(C₁₋₆-alkyl)₂ or onto which an aliphatic, heteroaliphatic, aromatic or heteroaromatic five- or six-membered ring is fused in the 2,3 positions and in the case of heteroaryl also in the 3,4 positions, with phenyl or heteroaryl also being able to contain further substituents.

A phenyl radical R₉ can, for example, correspond to the formula C

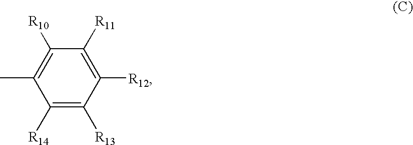

where
R₁₀ is C₁₋₄-alkyl, C₁₋₄-alkoxy, C₁₋₄-alkylthio, phenyl, phenoxy, benzyl, benzyloxy, C₁₋₄-fluoroalkyl, F, Cl, Br, I, OH or N(C₁₋₄-alkyl)₂,
R₁₁ is a hydrogen atom or independently has one of the meanings of R₁₀, or
R₁₀ and R₁₁ together form unsubstituted or C₁₋₄-alkyl-, C₁₋₄-alkoxy-, C₁₋₄-fluoroalkyl-, F— or Cl-substituted —CH═CH—CH═CH—, —N═CH—CH═CH—, —CH═N—CH═CH—, —CH═CH—N═CH—, —CH═N—CH═N—, ═CH—X₂—CH═, ═CH—X₂—CH₂—, —CH₂—X₂—CH₂—, —X₂—CH₂—CH═, —X₁—CH₂—CH₂—, —CH₂CH₂CH₂—, —CH₂CH₂CH₂CH₂—, —O—CH₂—O—, —O—CH₂CH₂—O—, —CH₂—CH₂—O—, —CH₂—CH₂CH₂—O—, —O—CH₂—CH₂— or —O—CH₂CH₂—CH₂—,
X₂ is O, S, N, NH or N—C₁₋₄-alkyl, and
R₁₂, R₁₃ and R₁₄ are each, independently of one another, a hydrogen atom, C₁₋₄-alkyl, C₁₋₄-alkoxy, C₁₋₄-fluoroalkyl, F, Cl or N(C₁₋₆-alkyl)₂.

In the desired substitution of the radical of the formula C, not only R₁₀ but also preferably R₁₂ and R₁₄ are substituents. Preferred examples of substituents are methyl, ethyl, n- and i-propyl, n-, i- and t-butyl, methoxy, ethoxy, n- and i-propoxy, n-, i- and t-butoxy, F, CF₃ and CF₂—CF₃.

A heteroaryl radical R₉ can, for example, correspond to the formula D or E,

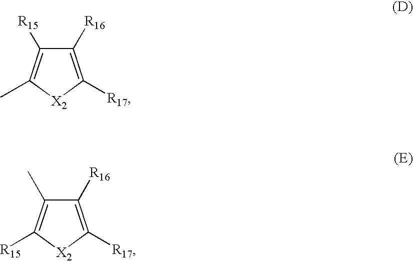

where
R₁₅ is C₁₋₄-alkyl, C₁₋₄-alkoxy, C₁₋₄-alkylthio, phenyl, phenoxy, benzyl, benzyloxy, C₁₋₄-fluoroalkyl, F, Cl or N(C₁₋₄-alkyl)₂,
R₁₆ and R₁₇ are each a hydrogen atom or independently have one of the meanings of R₁₅, or R₁₅ and R₁₆ in the formula D or R₁₆ and R₁₇ in each case together form unsubstituted or C₁₋₄-alkyl, C₁₋₄-alkoxy, C₁₋₄-fluoroalkyl, F- or Cl-substituted —CH═CH—CH═CH—, and
X₂ is O, S, N, NH or N—C₁₋₄-alkyl.

Preferred examples of substituents R₁₅, R₁₆ and R₁₇ are methyl, ethyl, n- and i-propyl, n-, i- and t-butyl, methoxy, ethoxy, n- and i-propoxy, n-, i- and t-butoxy, F, CF₃ and CF₂—CF₃.

The racemic aldehydes and ketones to be hydrogenated can correspond to the formula V,

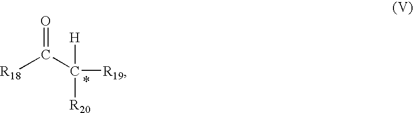

where
* denotes a stereogenic carbon atom,
R₁₈ is a hydrogen atom, a hydrocarbon radical or a C-bonded heterohydrocarbon radical having heteroatoms or heterogroups selected from the group consisting of O, S, N, NH and N(C₁-C₄-alkyl), each of which contains from 1 to 40, preferably from 1 to 30 and particularly preferably from 1 to 20, carbon atoms and is unsubstituted or substituted by radicals which are inert under the hydrogenation conditions,
R₁₉ and R₂₀ are each, independently of one another, a hydrocarbon radical or a C— or heteroatom-bonded heterohydrocarbon radical having heteroatoms or heterogroups selected from the group consisting of O, S, N, NH and N(C₁-C₄-alkyl), each of which contains from 1 to 40, preferably from 1 to 30 and particularly preferably from 1 to 20, carbon atoms and is unsubstituted or substituted by radicals which are inert under the hydrogenation conditions, or R₁₉ has this meaning and R₂₀ is halogen (F, Cl, Br and iodine), OH, SH or CN.
R₁₈ and R₂₀ together with the carbon atoms to which they are bound form a 3- to 10-membered hydrocarbon or heterohydrocarbon ring having heteroatoms or heterogroups selected from the group consisting of O, S, N, NH and N(C₁-C₄-alkyl), each of which is unsubstituted or substituted by radicals which are inert under the hydrogenation conditions, or
R₁₉ and R₂₀ together with the carbon atom to which they are bound form a 3- to 10-membered hydrocarbon or heterohydrocarbon ring having heteroatoms or heterogroups selected from the group consisting of O, S, N, NH and N(C₁-C₄-alkyl), each of which is unsubstituted or substituted by radicals which are inert under the hydrogenation conditions.

If at least two groups are to be hydrogenated in one process step, the inert radicals or substituents can contain unsaturated groups such as —C═C—, —C═N-(imine), —CH═O (aldehyde) or ═C═O (ketone). In this case, the radicals are not inert under the hydrogenation conditions.

R₁₉ and R₂₀ are different and cyclic radicals are unsubstituted or substituted, so that the choice of the radicals leads to a stereogenic carbon atom.

Suitable substituents are, for example, C₁-C₆-alkyl, C₂-C₆-alkenyl, C₁-C₆-alkoxy, C₁-C₆-haloalkyl, C₁-C₆-hydroxyalkyl, C₁-C₆-alkoxymethyl or C₁-C₆-alkoxyethyl, C₁-C₆-haloalkoxy, C₅-C₈-cycloalkyl, C₅-C₈-cycloalkoxy, C₅-C₈-cycloalkylmethyl or C₅-C₈-cycloalkylethyl, C₅-C₈-cycloalkoxy, C₅-C₈-cycloalkylmethoxy, phenyl, phenyloxy, benzyl, benzyloxy, phenylethyl, phenylethyloxy, halogen, —OH, —OR_{y}, ═O, —OC(O)R_{y}, —NH₂, —NHR_{y}, —NR_{y}R_{z}, —NH—C(O)—R_{y}, —NR_{y}—C(O)—R_{y}, —CO₂R_{y}, —CO₂—NH₂, —CO₂—NHR_{y}, —CO₂—NR_{y}R_{z}, where R_{y} and R_{z} are each, independently of one another, C₁-C₆-alkyl, cyclohexyl, cyclohexylmethyl, phenyl or benzyl.

The hydrocarbon radicals or heterohydrocarbon radicals can be substituted on one carbon atom and/or heteroatom and/or on different carbon atoms and/or heteroatoms by identical or different substituents, with a carbon atom also being able to be substituted by, for example, ═O and alkoxy or ═O and amino. In the latter case, R₁₉ and R₂₀ can, independently of one another, each be, for example, —CO₂R_{y}, —CO₂—NH₂, —CO₂—NHR_{y} or —CO₂—NR_{y}R_{z}. Heterohydrocarbon radicals bound via a heteroatom include, for example, oxy radicals (for example alkoxy, cycloalkoxy, phenoxy and benzyloxy), thio radicals (for example alkylthio) and amino radicals.

Preferred substituents are methyl, ethyl, n- and i-propyl, n- and t-butyl, vinyl, allyl, methyloxy, ethyloxy, n- and i-propyloxy, n- and t-butyloxy, trifluoromethyl, trichloromethyl, β-hydroxyethyl, methoxymethyl, ethoxymethyl, methoxyethyl or ethoxyethyl, trifluoromethoxy, cyclohexyl, cyclohexyloxy, cyclohexylmethyl, cyclohexylmethyloxy, phenyl, phenyloxy, benzyl, benzyloxy, phenylethyloxy, phenylethyl, halogen, —OH, —OR_{y}, —OC(O)R_{y}, —NH₂, —NHR_{y}, —NR_{y}R_{z}, —NH—C(O)—R_{y}, —NR_{y}—C(O)—R_{y}, —CO₂R_{y}, —CO₂—NH₂, —CO₂—NHR_{y}, —CO₂—NR_{y}R_{z}, where R_{y} and R_{z} are each, independently of one another, C₁-C₄-alkyl, cyclohexyl, cyclohexylmethyl, phenyl or benzyl.

Hydrocarbon radicals can each be a monovalent, saturated or unsaturated aliphatic radical having from 1 to 18 carbon atoms, a monovalent, saturated or unsaturated heteroaliphatic radical having from 1 to 17 carbon atoms and from 1 to 6, preferably from 1 to 4, heteroatoms, a saturated or unsaturated cycloaliphatic radical having from 4 to 8 ring carbons, a saturated or unsaturated heterocycloaliphatic radical having from 3 to 8 ring members and one or two heteroatoms from the group consisting of O, N, S and NR_{d}, a saturated or unsaturated cycloaliphatic-aliphatic radical having from 1 to 4 carbon atoms in the aliphatic group and from 4 to 8 ring carbons, a saturated or unsaturated heterocycloaliphatic-aliphatic radical having from 1 to 4 carbon atoms in the aliphatic group and from 3 to 7 carbon atoms and one or two heteroatoms from the group consisting of O, N, S and NR_{d} in the ring, an aromatic radical having from 6 to 10 carbon atoms, a heteroaromatic radical having from 4 to 9 carbon atoms and one or two heteroatoms from the group consisting of O, N, S and NR_{d} in the ring, an aromatic-aliphatic radical having from 6 to 10 carbon atoms in the aromatic ring and from 1 to 4 carbon atoms in the aliphatic group, or a heteroaromatic-aliphatic radical having from 4 to 9 carbon atoms and one or two heteroatoms from the group consisting of O, N, S and NR_{d} in the aromatic ring and from 1 to 4 carbon atoms in the aliphatic group, where R_{d} is H, C₁-C₈-alkyl, preferably C₁-C₄-alkyl, C₅- or C₆-cycloalkyl, C₆-C₁₀-aryl such as phenyl or naphthyl, C₇-C₁₂-aryl such as phenylmethyl or phenylethyl.

The aliphatic radical is preferably alkyl which may be linear or branched and preferably contains from 1 to 12, particularly preferably from 1 to 8, carbon atoms or preferably alkenyl or alkynyl, each of which may be linear or branched and preferably contains from 2 to 12, particularly preferably from 2 to 8, carbon atoms. The heteroaliphatic radical is preferably alkoxyl, alkylthio, primary and secondary amino having from 1 to 12 and preferably from 1 to 8 carbon atoms or alkoxyalkyl, alkylthioalkyl, aminoalkyl, alkoxyalkoxyl or aminoalkoxyl having from 1 to 6 and preferably from 1 to 4 carbon atoms in the alkyl groups or alkoxyl groups, from 1 to 12, particularly preferably from 1 to 8, carbon atoms in the alkoxy groups, alkylthio groups and in the primary or secondary amino groups. Examples of aliphatic and heteroaliphatic radicals are methyl, ethyl, n- and i-propyl. n-, i- and t-butyl, pentyl, i-pentyl, hexyl, heptyl, octyl, nonyl, decyl, undecyl, dodecyl, vinyl, allyl, ethynyl, propargyl, methoxy, ethoxy, n- and i-propoxy. n-, i- and t-butoxy, pentoxy, i-pentoxy, hexoxy, methylthio, methylamino, ethylamino, ethyl, n- and i-propylamino, n-, i- and t-butylamino, pentylamino, dimethylamino, diethylamino, methylethylamino, methoxymethyl, ethoxymethyl, methoxyethyl, methoxymethoxyl, ethoxymethoxyl, methoxyethoxyl, methylaminomethyl, dimethylaminomethyl, ethylaminomethyl, methylethylaminomethyl, dimethylaminoethyl, dimethylaminomethoxyl or dimethylaminoethoxyl and methoxyethylaminomethyl.

The cycloaliphatic radical is preferably cycloalkyl or cycloalkenyl having preferably from 3 to 8, particularly preferably 5 or 6, ring carbons. Some examples are cyclopropyl, cyclobutyl, cyclopentyl, cyclohexyl, cycloheptyl and cyclooctyl, and also cyclopentenyl, cyclohexenyl and cyclohexadienyl. Particular preference is given to cyclopentyl and cyclohexyl.

The heterocycloaliphatic radical is preferably heterocycloalkyl or heterocycloalkenyl having preferably from 3 to 6 carbon atoms, from 4 to 7 ring members and heteroatoms selected from the group consisting of —O—, S and —NR′—, where R′ is H, C₁-C₈-alkyl, preferably C₁-C₄-alkyl, C₅- or C₆-cycloalkyl, C₆-C₁₀-aryl such as phenyl or naphthyl, phenyl or phenylethyl. Some examples are pyrrolidinyl, pyrrolinyl, piperazinyl, N-methylpyrrolidinyl, tetrahydrofuranyl, dihydrofuranyl, tetrahydrothiophenyl and dihydrothiophenyl.

The heterocycloaliphatic-aliphatic radical is preferably heterocycloalkylalkyl or heterocycloalkylalkenyl having preferably from 3 to 6 carbon atoms, from 4 to 7 ring members and heteroatoms selected from the group consisting of —O—, S, N and —NR′—, and also from 1 to 4 or from 2 to 4 carbon atoms in the alkyl or alkenyl group, where R_{d} is H, C₁-C₈-alkyl, preferably C₁-C₄-alkyl, C₅- or C₆-cycloalkyl, C₆-C₁₀-aryl such as phenyl or naphthyl, phenyl or phenylethyl, and preferably from 1 to 4, particularly preferably 1 or 2, carbon atoms in the alkyl group or from 2 to 4 and particularly preferably 2 or 3 carbon atoms in the alkenyl group. Examples are pyrrolidinylmethyl, pyrrolidinylethyl or pyrrolidinylethenyl, pyrrolinylmethyl, pyrrolinylethyl or pyrrolinylethenyl, tetrahydrofuranylmethyl, tetrahydrofuranylethyl or tetrahydrofuranylethenyl, dihydrofuranylmethyl, dihydrofuranylethyl or dihydrofuranylethenyl, dihydrothiophenylmethyl or tetrahydrothiophenylmethyl and piperazinylmethyl, piperazinylethyl or piperazinylethenyl.

The aromatic radicals are preferably anthracenyl, phenanthrenyl, naphthyl and phenyl, with preference being given to phenyl and naphthyl.

The aromatic-aliphatic radicals are preferably phenyl- or naphthyl-C₁-C₄-alkyl or —C₂-C₄-alkenyl. Some examples are benzyl, naphthylmethyl, β-phenylethyl and β-phenylethenyl.

The heteroaromatic radicals are preferably 5- or 6-membered, fused or unfused ring systems. Some examples are pyridinyl, pyrimidinyl, pyrazinyl, pyrrolyl, furanyl, oxazolyl, thiophenyl, imidazolyl, benzofuranyl, indolyl, benzothiophenyl, benzimidazolyl, quinolinyl, isoquinolinyl, quinazolinyl, quinoxalinyl.

The heteroaromatic-aliphatic radicals are preferably 5- or 6-membered, fused or unfused ring systems which are bound via one of their carbon atoms or via an N atom to the free bond of an alkyl group or alkenyl group, with the alkyl group preferably having from 1 to 6, particularly preferably 1 or 4, carbon atoms and the alkenyl group preferably having from 2 to 6, particularly preferably from 2 to 4, carbon atoms. Some examples are pyridinylmethyl, pyridinylethyl or pyridinylethenyl, pyrimidinylmethyl, pyrimidinylethyl or pyrimidinylethenyl, pyrrolylmethyl, pyrrolylethyl or pyrrolylethenyl, furanylmethyl, furanylethyl or furanylethenyl, imidazolylmethyl, imidazolylethyl or imidazolylethenyl, thiophenylmethyl, thiophenylethyl or thiophenylethenyl, indolylmethyl, indolylethyl or indolylethenyl, benzofuranylmethyl, benzothiophenylmethyl and quinolinylmethyl or quinolinylethyl.

When R₁₉ and R₂₀ together form a hydrocarbon or heterohydrocarbon ring, R₁₉ and R₂₀ can together be C₂-C₇-alkylene or C₂-C₆-heteroalkylene or 1,2-phenylene-fused C₂-C₇-alkylene or C₂-C₆-heteroalkylene. Some examples are dimethylene to pentamethylene, —CH₂—O—, —CH₂—NH—, —CH₂—N(CH₃)—, —CH₂—CH₂—O—, —CH₂—CH₂—NH—, —CH₂—CH₂—N(CH₃)—, —CH₂—CH₂—O—CH₂—, —CH₂—CH₂—NH—CH₂—, —CH₂—CH₂—N(CH₃)—CH₂—, —CH₂—CH₂—O—CH₂—CH₂—, —CH₂—CH₂—NH—CH₂—CH₂—, —CH₂—CH₂—N(CH₃)—CH₂—CH₂—, —CH₂—CH₂—N═CH—CH₂—, —CH₂—CH₂—N(−)CH₂—CH₂—, —CH₂—CH₂—CH₂—CH₂—NH— and —CH₂—CH₂—CH₂—CH₂—O—.

When R₁₈ and R₂₀ together form a hydrocarbon or heterohydrocarbon ring, R₁₈ and R₂₀ can together be C₁-C₆-alkylene, C₂-C₅-heteroalkylene or alkylene or heteroalkylene fused with C₅-C₆-cycloalkylene, C₃-C₆-heterocycloalkylene, 1,2-phenylene, 1,2-naphthylene or heteroarylene having 5 or 6 ring members and 1 or 2 heteroatoms from the group consisting of O, N, S and NR_{d}, where R_{d} is as defined above. Examples of alkylene are dimethylene to hexamethylene. Examples of heteroalkylene have been given above for R₁₉ and R₂₀. Examples of fused alkylene or heteroalkylene are tetrahydronaphthylene, hexahydronaphthylene, octahydronaphthylene or decahydronaphthylene, tetrahydroquinolinylene, hexahydroquinolinylene, octahydroquinolinylene or decahydroquinolinylene, dihydrofuranylene, tetrahydrofuranylene, hexahydrofuranylene or octahydrofuranylene, dihydrothiophenylene, tetrahydrothiophenylene, hexahydrothiophenylene or octahydrothiophenylene, dihydroindolylene, tetrahydroindolylene, hexahydroindolylene or octahydroindolylene and dihydroindanylene, tetrahydroindanylene, hexahydroindanylene or octahydroindanylene.

In a preferred embodiment, the compounds of the formula V correspond to those of the formulae

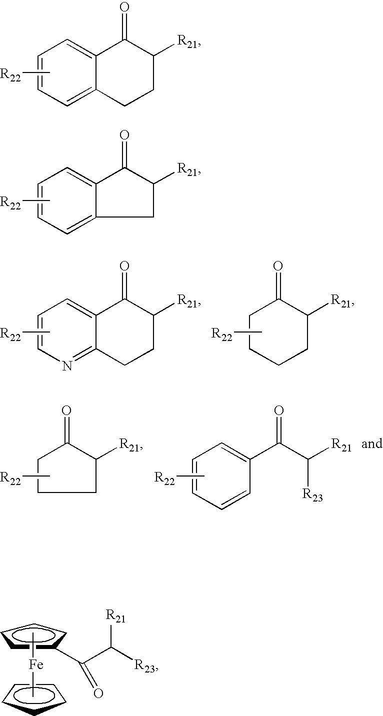

where
R₂₁ and R₂₃ are each, independently of one another, linear or branched C₁-C₆-alkyl (for example methyl, ethyl, n- and i-propyl, n-, i- and t-butyl), C₁-C₆-alkoxy (for example methoxy, ethoxy, n- and i-propoxy, n-, i- and t-butoxy), phenoxy, benzyloxy, —C(O)OC₁-C₆-alkyl (alkyl is preferably methyl or ethyl) or —C(O)—N(OC₁-C₆-alkyl)₂ (alkyl is preferably methyl or ethyl), and R₂₁ and R₂₃ are different, and
R₂₂ is hydrogen, C₁-C₄-alkyl (for example methyl or ethyl) or C₁-C₄-alkoxy (for example methoxy or ethoxy).

The process of the invention can, for example, be carried out by placing the catalyst together with the nitrogen base in an autoclave, if appropriate together with a solvent, then adding the racemic aldehyde or ketone, then displacing the air with an inert gas, for example noble gases, injecting hydrogen and then starting the reaction, if appropriate with stirring or shaking, and hydrogenating until no more hydrogen uptake is observed. The alcohols formed can be isolated and purified by customary methods, for example distillation, crystallization and chromatographic methods.

The alcohols which can be prepared according to the invention are valuable intermediates for preparing natural active compounds (B. T. Cho et al. in Tetrahedron: Asymmetry Vol. 5, No. 7 (1994), pages 1147 to 1150) and synthetic pharmaceutical active compounds and pesticides.

Furthermore, it has surprisingly been found that higher diastereomer ratios and higher enantiomeric excesses can be achieved when R₄ and R₅ in the formula II are identical phenyl radicals which are substituted in at least one ortho position relative to the P atom. It has also been found that such ligands in ruthenium complexes as catalysts in the hydrogenation of selected prochiral ketones can lead to considerably improved catalyst activities and higher enantioselectivities. Furthermore, an unexpectedly high stereoselectivity has been observed in the hydrogenation of prochiral ketones and chemoselectivity in the hydrogenation of ethylenically unsaturated ketones has been observed while using such hydrogenation catalysts.

The invention further provides compounds of the formula VI

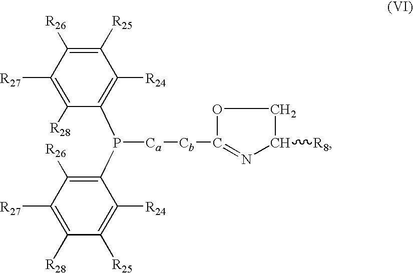

where
R₈ is C₁₋₆-alkoxy-C₁₋₄-alkyl, linear or branched C₁₋₈-alkyl, C₅₋₈-cycloalkyl, C₅₋₈-cycloalkyl-C₁₋₈-alkyl, C₆₋₁₄-aryl, C₇₋₁₂-aralkyl, C₃₋₁₂-heteroaryl, C₄₋₁₆-heteroaralkyl, where the cyclic radicals are unsubstituted or substituted by OH, F, Cl, Br, —CN, C₁₋₄-alkyl, C₁₋₄-alkoxy, trifluoromethyl, trifluoromethoxy, phenyl, C₁₋₄-alkyl or C₁₋₄-alkoxyphenyl, —C(O)O—C₁₋₄-alkyl or di(C₁-C₄-alkyl)amino,
Cₐ and C_{b} together are part of a five- or six-membered arene or heteroarene which is unsubstituted or substituted by F, Cl, Br, —CN, C₁₋₄-alkyl, C₁₋₄-alkoxy, trifluoromethyl, trifluoromethoxy, phenyl, C₁₋₄-alkylphenyl or C₁₋₄-alkoxyphenyl, —C(O)O—C₁₋₄-alkyl or di(C₁-C₄-alkyl)amino,
the radicals R₂₄ are each OH, F, Cl, Br, I, C₁₋₄-alkyl, C₁₋₄-alkoxy, trifluoromethyl, pentafluoroethyl, C₁₋₄-alkylthio, phenyl-C₁₋₄-alkyl, phenyl-C₁₋₄-alkoxy, phenyl-C₁₋₄-alkylthio or di(C₁-C₄-alkyl)amino, or
R₂₄ and R₂₅ together with the carbon atoms to which they are bound form a 5- or 6-membered hydrocarbon or heterohydrocarbon ring having heteroatoms or heterogroups selected from the group consisting of O, S, N, NH or N(C₁-C₄-alkyl), and
R₂₅ alone and R₂₆, R₂₇ and R₂₈ are each, independently of one another, a hydrogen atom, OH, F, Cl, Br, I, C₁₋₄-alkyl, C₁₋₄-alkoxy, trifluoromethyl, pentafluoroethyl, C₁₋₄-alkylthio or di(C₁-C₄-alkyl)amino.

The explanations and preferences described above apply to Cₐ and C_{b} and R₈. Cₐ and C_{b} are particularly preferably 1,2-phenylene, 1,2-naphthylene and 1,2-ferrocenylene. R₈ is particularly preferably C₁₋₄-alkyl (very particularly preferably i-propyl and t-butyl) and phenyl.

Preferred radicals R₂₄ are methyl, ethyl, n- and i-propyl, n-, i- and t-butyl, benzyl, benzyloxy, methoxy, ethoxy, n- and i-propoxy. Particular preference is given to methyl, methoxy and i-propoxy.

When R₂₄ and R₂₅ together with the carbon atoms to which they are bound form a ring, R₂₄ and R₂₅ together can be, for example, —CH═CH—CH═CH—, —N═CH—CH═CH—, —CH═N—CH═CH—, —CH═CH—N═CH—, —CH═N—CH═N—, ═CH—X₂—CH═, ═CH—X₂—CH₂—, —CH₂—X₂—CH₂—, —X₂—CH₂—CH═, —X₁—CH₂—CH₂—, —CH₂CH₂CH₂—, —CH₂CH₂CH₂CH₂—, —O—CH₂—O—, —O—CH₂CH₂—O—, —CH₂—CH₂—O—, —CH₂—CH₂CH₂—O—, —O—CH₂—CH₂— or —O—CH₂CH₂—CH₂—. Particular preference is given to R₂₄ and

R₂₅ together being —CH═CH—CH═CH—.

In a preferred embodiment, the ligands of the formula VI preferably correspond to the formula VIII,

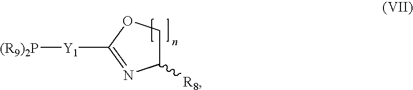

where
Y₁ is a radical of the formula (A) or (B):

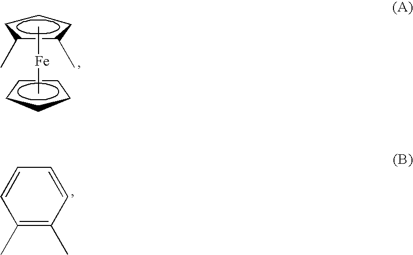

n is 1,
R₈ is linear or branched C₁₋₄-alkyl or phenyl and
R₉ is in each case phenyl which is substituted in the ortho position relative to the P/C bond by C₁₋₄-alkyl or C₁₋₄-alkoxy or R₉ is in each case 1-naphthyl.

The preparation of ligands used according to the invention can be carried out by methods known per se and analogous methods (see, for example, S. Uemura et al. in J. Organometallic Chem., 1999, 572,163 or WO 2004/050585) and is illustrated in the examples.

The invention also provides ruthenium complexes having ligands of the formula VI and corresponding to the formula VIII,

[(X)₂Ru(PR₁R₂R₃)(P-Z-N)] (VIII),

where
X is hydride, halide, C₁₋₈-alkoxide or C₁₋₈-acyloxy, P-Z-N is a compound of the formula VI or of the formula VII and R₁, R₂ and R₃ are as defined above.

The preferences indicated above apply to X, compounds of the formula VI and R₁, R₂ and R₃.

X is particularly preferably Cl or Br, R₁, R₂ and R₃ are each particularly preferably phenyl, and P-Z-N is particularly preferably a compound of the formula VIII.

The invention further provides for the use of the ruthenium complexes of the formula VIII for the hydrogenation of aldehydes and preferably prochiral ketones by means of hydrogen.

The following examples illustrate the invention. For comparative purposes, results of catalytic hydrogenations carried out using the known complex K_{comparison} (K_{comp}) (see, for example, S. Uemura et al. in Organometallics, 1999, 18, 2291 or WO 2004/050585):

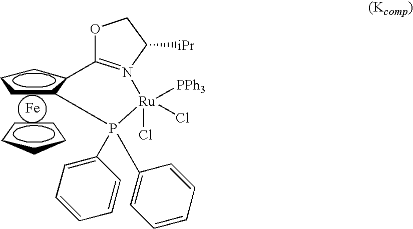

are also reported.

### A) Preparation of Novel P-Z-N Ligands

### EXAMPLE A1

### Preparation of (S)-4-isopropyl-2-[(S)-2-(bis(1-naphthyl)phosphino)ferrocen-1-yl]oxazoline of the Formula (Ligand L1)

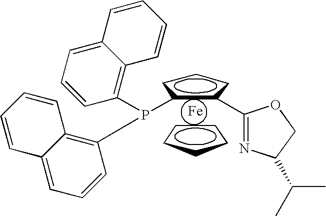

10 g (33.65 mmol) of (S)-4-isopropyl-2-(ferrocen-1-yl)oxazoline are dissolved in 100 ml of diethyl ether and cooled to −73° C. under argon. 31 ml (40.38 mmol, 1.2 equivalents) of s-butyl-Li (1.3M in cyclohexane) are added dropwise at this temperature and the mixture is stirred for 2 hours. 13.4 g (40.38 mmol, 1.2 equivalents) of bis(1-naphthyl)phosphine chloride are added all at once and the reaction mixture is warmed to room temperature over a period of 10 hours. The reaction is stopped by addition of 35 ml of saturated sodium hydrogencarbonate solution. The organic phase is separated off, washed with water, dried over sodium sulphate and the solvent is then removed. The crude product is then chromatographed on silica gel (eluent=3:1 heptane:methyl t-butyl ether [MTBE]). This gives 2.05 g (10.5% of theory) of the title compound as a yellow solid. ¹H NMR (C₆D₆, 300 MHz, ppm): 0.73 (d, 3H); 0.86 (d, 3H); 1.50 (m, 1H); 3.30-3.84 (m, 4H); 4.02 (s, 5H); 5.14 (b, 6H); 6.80-7.75 (m, 12H); 8.68 (b, 1H); 9.80 (b, 1H). ³¹P NMR (C₆D₆, 121.5 MHz, ppm): −39.3 (s).

### EXAMPLE A2

### Preparation of (S)-4-isopropyl-2-[(S)-2-(bis(2-methoxyphenyl)phosphino)-ferrocen-1-yl]oxazoline of the Formula (Ligand L2)

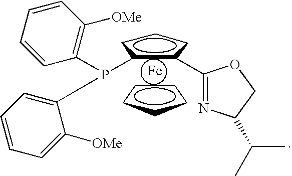

8 g (26.92 mmol) of (S)-4-isopropyl-2-(ferrocen-1-yl)oxazoline are dissolved in 100 ml of diethyl ether and cooled to −73° C. under argon. 25 ml (32.30 mmol, 1.2 equivalents) of s-butyl-Li (1.3M in cyclohexane) are added dropwise at this temperature and the mixture is stirred for 2 hours. 9 g (32.30 mmol, 1.2 equivalents) of bis(2-methoxyphenyl)phosphine chloride are added all at once and the reaction mixture is warmed to room temperature over a period of 10 hours. The reaction is stopped by addition of 35 ml of saturated sodium hydrogencarbonate solution. The organic phase is separated off, washed with water, dried over sodium sulphate and the solvent is then removed. The crude product is chromatographed on silica gel (eluent=3:1 heptane:MTBE). This gives 4.6 g (32% of theory) of the title compound as a yellow solid. ¹H NMR (C₆D₆, 300 MHz, ppm): 0.79 (d, 3H); 0.93 (d, 3H); 1.55 (m, 1H); 3.16 (s, 3H); 3.46 (s, 3H); 3.53 (t, 1H); 3.63 (m, 1H); 3.80-3.86 (m, 2H); 4.08 (b, 1H); 4.28 (s, 5H); 5.17 (b, 1H); 6.40-7.33 (m, 8H). ³¹P NMR (C₆D₆, 121.5 MHz, ppm): −41.4 (s).

### EXAMPLE A3

### Preparation of (S)-4-isopropyl-2-[(S)-2-(bis(2-methylphenyl)phosphino)ferrocen-1-yl]oxazoline of the Formula (Ligand L3)

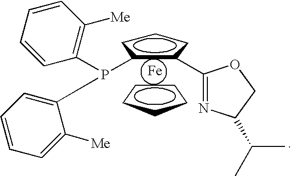

8 g (26.92 mmol) of (S)-4-isopropyl-2-(ferrocen-1-yl)oxazoline are dissolved in 100 ml of diethyl ether and cooled to −73° C. under argon. 25 ml (32.30 mmol, 1.2 equivalents) of s-butyl-Li (1.3M in cyclohexane) are added dropwise at this temperature and the mixture is stirred for 2 hours. 8 g (32.30 mmol, 1.2 equivalents) of bis(2-methylphenyl)phosphine chloride are added all at once and the reaction mixture is warmed to room temperature over a period of 10 hours. The reaction is stopped by addition of 35 ml of saturated sodium hydrogencarbonate solution. The organic phase is separated off, washed with water, dried over sodium sulphate and the solvent is then removed. The crude product is chromatographed on silica gel (eluent=3:1 heptane:MTBE). This gives 9.3 g (68% of theory) of the title compound as a yellow foam. The product still contains about 15% of the (S_{c},Rₚ)-diastereomer. ¹H NMR (C₆D₆, 300 MHz, ppm): 0.78 (d, 3H); 0.94 (d, 3H); 1.53 (m, 1H); 2.34 (s, 3H); 2.91 (s, 3H); 3.43 (t, 1H), 3.54-3.84 (m, 3H); 4.04 (b, 1H); 4.16 (s, 5H); 5.11 (b, 1H); 6.80-7.40 (m, 8H). ³¹P NMR (C₆D₆, 121.5 MHz, ppm): −35.0 (s).

### B) Preparation of Metal Complexes

### EXAMPLE B1

### In-Situ Preparation of [(Cl)₂Ru(Pphenyl₃)(ligand L1)], K1

The ruthenium compound [RuCl₂(PPh₃)₃] (4.81 mg, 5.02 μmol) and ligand L1 (2.95 mg, 5.07 μmol) are admixed with 3 ml of toluene under argon in a Schlenk tube. The mixture is stirred at 90° C. for 2 hours, resulting in formation of a brown solution. This is used directly for the hydrogenation.

### EXAMPLE B2

### Preparation of [(Cl)₂Ru(Pphenyl₃) (ligand L2)], K₂

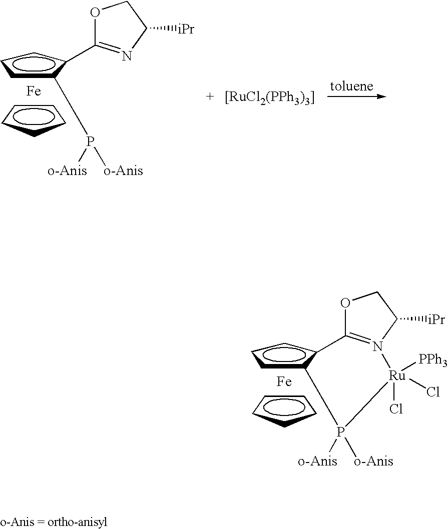

272.5 mg (0.284 mmol) of dichlorotris(triphenylphosphine)ruthenium(II) and 160.0 mg (0.296 mmol) of ligand L2 are placed in a 10 ml Schlenk tube and admixed with 6.5 ml of dry toluene under argon. The dark suspension is stirred overnight at room temperature, resulting in a colour change to orange-red. After addition of 4 ml of dry pentane, the stirrer is switched off and the supernatant orange solution is filtered off with suction from the orange solid. The solid is washed five times with 4 ml each time of pentane and dried in a high vacuum. This gives 244 mg (88% of theory) of the title compound as an orange powder. ³¹P-NMR (C₆D₆, 121.5 MHz, ppm): 49.7 (d, J=40), 61.2 (d, J=40).

### EXAMPLE B3

### Preparation of [(Cl)₂Ru(Pphenyl₃)(ligand L3)], K₃

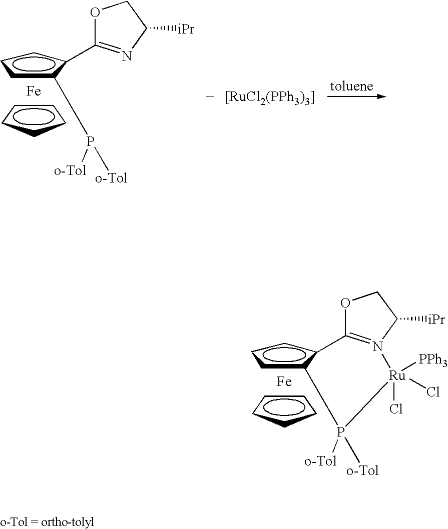

278.7 mg (0.291 mmol) of dichlorotris(triphenylphosphine)ruthenium(II) and 154.0 mg (0.302 mmol) of ligand L3 are placed in a 10 ml Schlenk tube and admixed with 6.0 ml of dry toluene under argon. The dark suspension is stirred overnight at room temperature, resulting in a colour change to red. After addition of 4 ml of dry pentane, the stirrer is switched off and the supernatant solution is separated off from the red solid. The solid is washed three times with 4 ml each time of pentane and dried in a high vacuum. This gives 316 mg (36% m/m of toluene, effective yield: 73% of theory) of the title compound as a red powder. ¹H-NMR (CD₂Cl₂, 300.1 MHz, ppm, 2 forms, main and secondary, in a ratio of 87:13): 0.67 (d, J=7.0, CHCH₃), 0.82 (d, J=7.0, CHCH₃), 1.79 (d, J_{PH}=5.9, CH₃), 2.05 (s, CH₃), 3.04 (sₑₚₜd, J=7.0, 2.8, CHCH₃), 3.13 (dd, J=9.6, 8.6, CHH), 3.51 (s, C₅H₅), 3.92 (dd, J=8.6, 3.7, CHH), 4.53 (dt, J=9.7, 3.1, NCH), 4.57 (t, J=2.6, 1 Cp-H), 4.65 (m, 1 Cp-H), 4.66 (m, 1 Cp-H), 6.11-6.18 (m, 1 Ar—H), 6.43-6.52 (m, 1 Ar—H), 6.91-7.76 (m, 21 Ar—H). ³¹P-NMR (C₆D₆, 121.5 MHz, ppm): 51.4 (d, J=38), 62.4 (d, J=38). ³¹P-NMR (CD₂Cl₂, 121.5 MHz, ppm): 49.0 (d, J=36, secondary), 52.4 (d, J=39, main), 58.5 (d, J=36, secondary), 61.1 (d, J=39, main).

### C) Hydrogenation of Prochiral Ketones

### General Method:

In a Schlenk tube, the metal complex K₁, K₂ or K₃ or K_{comp} for comparison (hydrogenation catalyst) is dissolved in 1 ml of absolute toluene (method A) or freshly distilled iso-propanol (method B). As an alternative, the solution of a ruthenium complex generated in situ can also be used. The starting material is dissolved in 2 ml of toluene (method A) or 1 ml of iso-propanol (method B) in a second Schlenk tube. The contents of the two Schlenk tubes are transferred under argon pressure by means of a Teflon tube into a 50 ml autoclave which has previously been filled with argon. Finally, 1 ml of aqueous, degassed NaOH (1M, method A) or 1 ml of a 1M solution of potassium t-butoxide in isopropanol (method B) is added. The autoclave is closed, the contents are flushed three times with argon and then three times with hydrogen and the hydrogen pressure is then set. If desired, the autoclave is heated by means of an oil bath. After the reaction temperature has been reached, the pressure is set again and the magnetic stirrer is started. After the desired hydrogenation time, the stirring and heating is switched off and the autoclave is ventilated with argon after cooling. A sample of the crude product is examined by means of GC and HPLC analysis.

Method A: base: 1.0 ml (1.0 mmol) of 1M aqueous NaOH; solvent: toluene, 3 ml.

Method B: base: 1.0 ml (1.0 mmol) of 1M potassium t-butoxide; solvent: 2-propanol, 2 ml. S/C is the ratio of substrate to catalyst. Solv. is solvent.

### EXAMPLES C1 AND C2

### Hydrogenation of α,α,α-trifluoroacetophenone

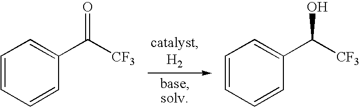

C1: α,α,α-Trifluoroacetophenone: 0.14 ml (1.0 mmol); Ru compound: [RuCl₂(PPh₃)₃] 4.81 mg (5.02 μmol); ligand L1: 2.95 mg (5.07 μmol); S/C: 200; base: 1.0 ml (1.0 mmol) of 1M aqueous NaOH; solvent: toluene 3 ml; 80 bar of H₂/25° C.; 18 hours. 100% conversion, 81% ee.

C2 (comparison): α,α,α-Trifluoroacetophenone: 0.14 ml (1.0 mmol); catalyst: 4.58 mg (5.0 μmol) of K_{comp}; S/C: 200; base: 1.0 ml (1.0 mmol) of 1M aqueous NaOH; solvent: toluene 3 ml; 80 bar of H₂/25° C.; 16 hours. 100% conversion, only 21% ee.

### EXAMPLES C3, C4 AND C5

### Hydrogenation of 2-trifluoromethylacetophenone

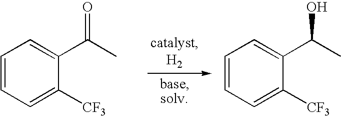

C3: 2-Trifluoromethylacetophenone: 0.15 ml (1.0 mmol); Ru compound [RuCl₂(PPh₃)]: 4.79 mg (5.0 μmol); ligand L1: 2.91 mg (5.0 μmol); S/C: 200; base: 1.0 ml (1.0 mmol) of 1M aqueous NaOH; solvent: toluene 3 ml; 80 bar of H₂/25° C.; 16 hours. 100% conversion, 96% ee (R).

C4: 2-Trifluoromethylacetophenone: 0.15 ml (1.0 mmol); catalyst: 4.88 mg (5.00 μmol) of K₂; S/C: 200; base: 1.0 ml (1.0 mmol) of 1M aqueous NaOH; solvent: toluene 3 ml; 80 bar of H₂/25° C.; 17 hours. 100% conversion, 94% ee (R).

C5 (comparison): 2-Trifluoromethylacetophenone: 0.15 ml (1.0 mmol); catalyst: 4.58 mg (5.00 μmol) of K_{comp}; S/C: 200; base: 1.0 ml (1.0 mmol) of 1M aq. NaOH; solvent: toluene 3 ml; 80 bar of H₂/25° C.; 16 hours. 100% conversion, 90% ee (R).

### EXAMPLE C6

### Hydrogenation of 2,4-dichloroacetophenone

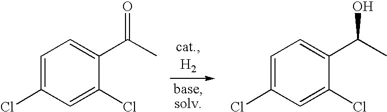

2,4-Dichloroacetophenone: 189 mg (1 mmol); Ru compound [RuCl₂(PPh₃)]: 4.81 mg (5.02 μmol); ligand L1: 2.95 mg (5.07 μmol); S/C: 200; base: 1.0 ml (1.0 mmol) of 1M aqueous NaOH; solvent: toluene 3 ml; 80 bar of H₂/25° C.; 18 hours. 100% conversion, 97% ee.

### EXAMPLES C7 AND C8

### Hydrogenation of Indanone

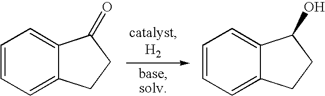

C7: Indanone: 132.7 mg (1.0 mmol); catalyst: 4.90 mg (5.02 μmol) of K₂; S/C: 200; base: 1.0 ml (1.0 mmol) of 1M potassium t-butoxide; solvent: 2 ml of 2-propanol; 80 bar of H₂/25° C.; 20 hours. 100% conversion, 84% ee.

C8: Indanone: 132.7 mg (1.0 mmol); Ru compound [RuCl₂(PPh₃)]: 4.81 mg (5.02 μmol); ligand L1: 2.95 mg (5.07 μmol); S/C: 200; base: 1.0 ml (1.0 mmol) of 1M aqueous NaOH; solvent: toluene 3 ml; 80 bar of H₂/25° C.; 18 hours. 94% conversion, 90% ee.

### D) Hydrogenation of Chiral Ketones

### EXAMPLE D1

### Hydrogenation of 2-methyl-1-tetralone

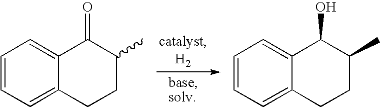

2-Methyl-1-tetralon (racemic): 320 mg (2 mmol); catalyst: 0.02 mmol of K₂; S/C: 100; base: potassium t-butoxide: 112 mg; solvent: ethanol/toluene (1:1) 5 ml; 80 bar of H₂/25° C.; 20 hours. 97% conversion, diastereomer ratio: cis/trans=99:1; 94.7% ee (cis).

### EXAMPLE D2

### Hydrogenation of 2-methoxycyclohexanone

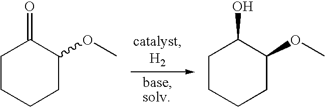

2-Methoxycyclohexanone: 256 mg (2 mmol); catalyst: 0.02 mmol of K₂; S/C: 100; base:

potassium t-butoxide: 112 mg; solvent: ethanol/toluene (1:3) 8 ml; 80 bar of H₂/25° C.; 20 hours. 100% conversion, diastereomer ratio: 27:73; 58% or 73% ee, respectively.

### EXAMPLE D3

### Hydrogenation of 2-methyl-1-indanone

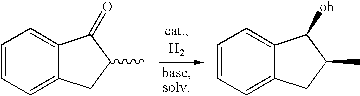

2-Methyl-1-indanone (racemic): 0.14 ml (1.0 mmol); catalyst: 4.90 mg (5.02 μmol) of K₂; S/C: 200; base: 1.0 ml (1.0 mmol) of 1M aqueous NaOH; solvent: toluene 3 ml; 80 bar of H₂/25° C.; 18 hours. 93% conversion, diastereomer ratio: 95:5; 82% or 39% ee, respectively.

### E) Hydrogenation of Enones

### Comparison

### EXAMPLES E1 AND E2

### Hydrogenation of 3-methyl-2-cyclohexenone

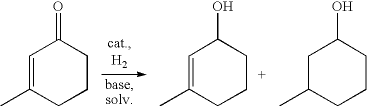

E1: 3-Methyl-2-cyclohexenone: 0.11 ml (0.97 mmol); catalyst: 4.90 mg (5.02 μmol) of K₂; S/C: 193; base: 1.0 ml (1.0 mmol) of 1M aqueous NaOH; solvent: toluene 3 ml; 80 bar of H₂/25° C.; 18 hours. 97% conversion, selectivity: 93% of 3-methyl-2-cyclohexen-1-ol (5% ee) and 4% of 3-methylcyclohexan-1-ol.

E2 (comparison): 3-Methyl-2-cyclohexenone: 0.11 ml (0.97 mmol); catalyst: 4.60 mg (5.02 μmol) of K_{comp}; S/C: 193; base: 1.0 ml (1.0 mmol) of 1M aqueous NaOH; solvent: toluene 3 ml; 80 bar of H₂/25° C.; 18 hours. 100% conversion, selectivity: 100% of 3-methylcyclohexan-1-ol, d.r.: 72:28.

1. Process for preparing a predominantly enantiomeric primary alcohol or a predominantly diastereomeric secondary alcohol by reacting aldehydes or ketones with hydrogen in the presence of a base and a ruthenium complex containing a bidentate ligand having coordinating P and N atoms, a monophosphine ligand and anionic and/or uncharged ligands as homogeneous catalyst, with the charge being balanced by one or two monovalent acid anions or a divalent acid anion when uncharged ligands are present, characterized in that a racemic aldehyde or ketone having a stereogenic carbon atom in the α position relative to the C(O) group and containing the structural element —(O)C—C—CH— is reacted.

2. Process according to claim 1, characterized in that the ruthenium complex corresponds to the general formula I,
[XYRu(PR₁R₂R₃)(P-Z-N)] (I),
where
X and Y are each, independently of one another, a hydride, halide, C₁₋₈-alkoxide or C₁₋₈-acyloxy or a coordinated organic solvent ligand containing at least one heteroatom from the group consisting of O, S and N, with a resulting cationic complex having one or two solvent ligands being neutralized by one or two monovalent anions or a divalent anion,
R₁, R₂ and R₃ are each, independently of one another, a hydrocarbon radical or a C-bonded heterohydrocarbon radical having heteroatoms selected from the group consisting of O, S, N, NH and N(C₁-C₄-alkyl), each of which has from 1 to 22, preferably from 1 to 14 and particularly preferably from 1 to 10, carbon atoms and is unsubstituted or substituted, or one of the radicals R₁, R₂, R₃ is as defined above and the remaining two radicals together with the phosphorus atom and carbon atoms form a 4- to 8-membered, unsubstituted or substituted ring, and
P-Z-N is a bidentate ligand of the formula (II), 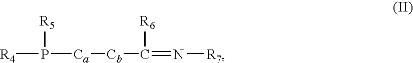
where
R₄ and R₅ are each, independently of one another, a hydrocarbon radical or a C-bonded heterohydrocarbon radical having heteroatoms selected from the group consisting of O, S, N, NH or N(C₁-C₄-alkyl), each of which has from 1 to 22, preferably from 1 to 14 and particularly preferably from 1 to 10, carbon atoms, or R₄ and R₅ together with the phosphorus atom and further carbon atoms form a 4- to 8-membered ring, with R₄ and R₅ being unsubstituted or substituted,
Cₐ and C_{b} together are part of a five- or six-membered arene or heteroarene which is unsubstituted or substituted by OH, F, Cl, Br, —CN, C₁₋₄-alkyl, C₁₋₄-alkoxy, trifluoromethyl, trifluoromethoxy, phenyl, C₁₋₄-alkyl or C₁₋₄-alkoxyphenyl, —C(O)O—C₁₋₄-alkyl or di(C₁-C₄-alkyl)amino,
R₆ is a hydrogen atom, a linear, branched or cyclic C₁₋₁₀-alkyl or C₂₋₁₀-alkenyl group or a C₆₋₁₀-aryl group, each of which is unsubstituted or substituted by C₁₋₄-alkyl, C₁₋₄-alkoxy, trifluoromethyl, trifluoromethoxy, di(C₁-C₄-alkyl)amino, phenyl, benzyl, C₁₋₄-alkylphenyl or C₁₋₄-alkylbenzyl, or R₆ is a —OR_{6′} or —NR_{6′}R_{6″} radical, where R_{6′} and R_{6″} have the same meanings as R₆,
R₇ is a hydrogen atom, a linear, branched or cyclic C₁₋₁₀-alkyl or C₂₋₁₀-alkenyl group, or R₇ is a R_{7′}CO— or R_{7′}SO₂— radical, where R_{7′} is a hydrocarbon radical having from 1 to 14 carbon atoms, or
R₆ and R₇ together with the group —C═N— form an unsaturated five- to ten-membered, preferably five- to seven-membered, substituted or unsubstituted heterocycle.

3. Process according to claim 2, characterized in that the ligands of the formula II correspond to the formula IV, 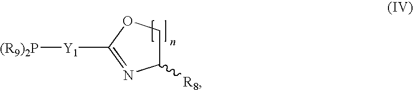
where
Y₁ is a ferrocene radical of the formula (A) which may be unsubstituted or be substituted by C₁₋₄-alkyl or halogen, or a radical of the formula (B) which may be unsubstituted or be substituted by OH, F, Cl, Br, —CN, C₁₋₄-alkyl, C₁₋₄-alkoxy, trifluoromethyl, trifluoromethoxy, phenyl, C₁₋₄-alkylphenyl or C₁₋₄-alkoxyphenyl, —C(O)O—C₁₋₄-alkyl or di(C₁-C₄-alkyl)amino: 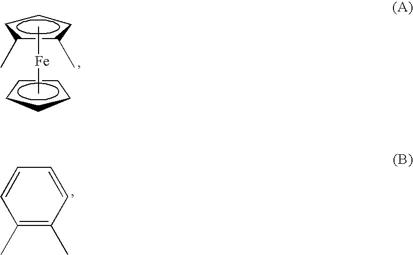
n is 1 or 2, preferably 1,
R₈ is C₁₋₄-alkoxy-C₁₋₂-alkyl, linear or branched C₁₋₄-alkyl, cyclohexyl, phenyl or benzyl, where the cyclic radicals may be substituted as defined above, and
the radicals R₉ are each phenyl or C-bonded heteroaryl having heteroatoms selected from the group consisting of O, S, N, NH or N—C₁₋₄-alkyl, each of which is either substituted in at least one ortho position relative to the P—C bond by C₁₋₆-alkyl, C₁₋₆-alkoxy, C₁₋₆-alkylthio, phenyl, phenoxy, benzyl, benzyloxy, C₁₋₆-fluoroalkyl, F, Cl, Br, OH or N(C₁₋₆-alkyl)₂ or onto which an aliphatic, heteroaliphatic, aromatic or heteroaromatic five- or six-membered ring is fused in the 2,3 positions and in the case of heteroaryl also in the 3,4 positions, with phenyl or heteroaryl also being able to contain further substituents.

4. Process according to claim 3, characterized in that a phenyl radical R₉ corresponds to the formula C, 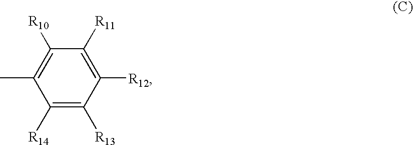
where
R₁₀ is C₁₋₄-alkyl, C₁₋₄-alkoxy, C₁₋₄-alkylthio, phenyl, phenoxy, benzyl, benzyloxy, C₁₋₄-fluoroalkyl, F, Cl, Br, I, OH or N(C₁₋₄-alkyl)₂,
R₁₁ is a hydrogen atom or independently has one of the meanings of R₁₀, or
R₁₀ and R₁₁ together form unsubstituted or C₁₋₄-alkyl-, C₁₋₄-alkoxy-, C₁₋₄-fluoroalkyl-, F— or Cl-substituted —CH═CH—CH═CH—, —N═CH—CH═CH—, —CH═N—CH═CH—, —CH═CH—N═CH—, —CH═N—CH═N—, ═CH—X₂—CH═, ═CH—X₂—CH₂—, —CH₂—X₂—CH₂—, —X₂—CH₂—CH═, —X₁—CH₂—CH₂—, —CH₂CH₂CH₂—, —CH₂CH₂CH₂CH₂—, —O—CH₂—O—, —O—CH₂CH₂—O—, —CH₂—CH₂—O—, —CH₂—CH₂CH₂—O—, —O—CH₂—CH₂— or —O—CH₂CH₂—CH₂—,
X₂ is O, S, N, NH or N—C₁₋₄-alkyl, and
R₁₂, R₁₃ and R₁₄ are each, independently of one another, a hydrogen atom, C₁₋₄-alkyl, C₁₋₄-alkoxy, C₁₋₄-fluoroalkyl, F, Cl or N(C₁₋₆-alkyl)₂.

5. Process according to claim 1, characterized in that it is carried out at a pressure of from 1·10⁵ to 1·10⁷ Pa (from 1 to 100 bar).

6. Process according to claim 1, characterized in that it is carried out at a temperature of from 0° C. to 100° C.

7. Process according to claim 1, characterized in that it is carried out in the presence of a solvent or a mixture of at least two solvents.

8. Process according to claim 1, characterized in that inorganic bases selected from the group consisting of alkaline earth metal or alkali metal hydroxides, alkali metal alkoxides, alkali metal carbonates or hydrogencarbonates and alkali metal amides or organic nitrogen bases are used as bases.

9. Process according to claim 1, characterized in that the molar ratio of base to substrate is from 10 to 0.1.

10. Process according to claim 1, characterized in that the molar ratio of substrate to ruthenium complex is from 106 to 20.

11. Process according to claim 1, characterized in that the racemic aldehydes and ketones to be hydrogenated correspond to the formula V, 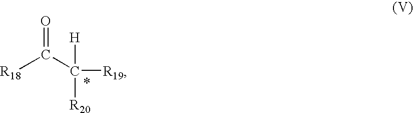
where
* denotes a stereogenic carbon atom,
R₁₈ is a hydrogen atom, a hydrocarbon radical or a C-bonded heterohydrocarbon radical having heteroatoms or heterogroups selected from the group consisting of O, S, N, NH and N(C₁-C₄-alkyl), each of which contains from 1 to 40, preferably from 1 to 30 and particularly preferably from 1 to 20, carbon atoms and is unsubstituted or substituted by radicals which are inert under the hydrogenation conditions or by radicals which contain unsaturated groups —C═C—, —C═N-(imine), —CH═O (aldehyde) or ═C═O (ketone),
R₁₉ and R₂₀ are each, independently of one another, a hydrocarbon radical or a C— or heteroatom-bonded heterohydrocarbon radical having heteroatoms or heterogroups selected from the group consisting of O, S, N, NH and N(C₁-C₄-alkyl), each of which contains from 1 to 40, preferably from 1 to 30 and particularly preferably from 1 to 20, carbon atoms and is unsubstituted or substituted by radicals which are inert under the hydrogenation conditions, or
R₁₉ has this meaning and R₂₀ is halogen, OH, SH or CN;
R₁₈ and R₂₀ together with the carbon atoms to which they are bound form a 3- to 10-membered hydrocarbon or heterohydrocarbon ring having heteroatoms or heterogroups selected from the group consisting of O, S, N, NH and N(C₁-C₄-alkyl), each of which is unsubstituted or substituted by radicals which are inert under the hydrogenation conditions, or by radicals which contain unsaturated groups —C═C—, —C═N-(imine), —CH═O (aldehyde) or ═C═O (ketone), or
R₁₉ and R₂₀ together with the carbon atom to which they are bound form a 3- to 10-membered hydrocarbon or heterohydrocarbon ring having heteroatoms or heterogroups selected from the group consisting of O, S, N, NH and N(C₁-C₄-alkyl), each of which is unsubstituted or substituted by radicals which are inert under the hydrogenation conditions or by radicals which contain unsaturated groups —C═C—, —C═N-(imine), —CH═O (aldehyde) or ═C═O (ketone).

12. Compounds of the formula VI, 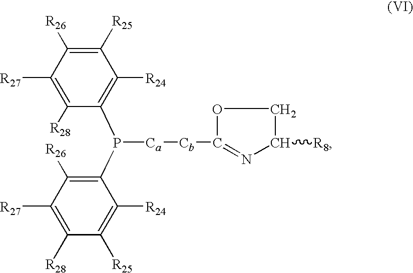
where
R₈ is C₁₋₆-alkoxy-C₁₋₄-alkyl, linear or branched C₁₋₈-alkyl, C₅₋₈-cycloalkyl, C₅₋₈-cycloalkyl-C₁₋₈-alkyl, C₆₋₁₄-aryl, C₇₋₁₂-aralkyl, C₃₋₁₂-heteroaryl, C₄₋₁₆-heteroaralkyl, where the cyclic radicals are unsubstituted or substituted by OH, F, Cl, Br, —CN, C₁₋₄-alkyl, C₁₋₄-alkoxy, trifluoromethyl, trifluoromethoxy, phenyl, C₁₋₄-alkyl or C₁₋₄-alkoxyphenyl, —C(O)O—C₁₋₄-alkyl or di(C₁-C₄-alkyl)amino,
Cₐ and C_{b} together are part of a five- or six-membered arene or heteroarene which is unsubstituted or substituted by F, Cl, Br, —CN, C₁₋₄-alkyl, C₁₋₄-alkoxy, trifluoromethyl, trifluoromethoxy, phenyl, C₁₋₄-alkylphenyl or C₁₋₄-alkoxyphenyl, —C(O)O—C₁₋₄-alkyl or di(C₁-C₄-alkyl)amino,
the radicals R₂₄ are each OH, F, Cl, Br, I, C₁₋₄-alkyl, C₁₋₄-alkoxy, trifluoromethyl, pentafluoroethyl, C₁₋₄-alkylthio, phenyl-C₁₋₄-alkyl, phenyl-C₁₋₄-alkoxy, phenyl-C₁₋₄-alkylthio or di(C₁-C₄-alkyl)amino, or
R₂₄ and R₂₅ together with the carbon atoms to which they are bound form a 5- or 6-membered hydrocarbon or heterohydrocarbon ring having heteroatoms or heterogroups selected from the group consisting of O, S, N, NH or N(C₁-C₄-alkyl), and
R₂₅ alone and R₂₆, R₂₇ and R₂₈ are each, independently of one another, a hydrogen atom, OH, F, Cl, Br, I, C₁₋₄-alkyl, C₁₋₄-alkoxy, trifluoromethyl, pentafluoroethyl, C₁₋₄-alkylthio or di(C₁-C₄-alkyl)amino.

13. Compounds according to claim 12, characterized in that they correspond to the formula VIII, 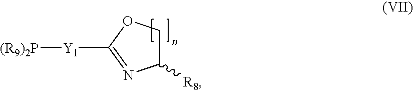
where
Y₁ is a radical of the formula (A) or (B): 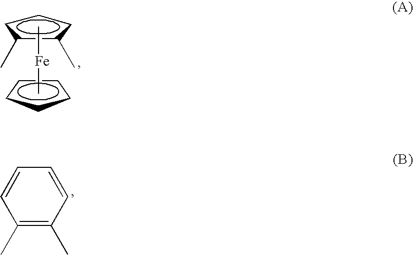
n is 1,
R₈ is linear or branched C₁₋₄-alkyl or phenyl and
R₉ is in each case phenyl which is substituted in the ortho position relative to the P/C bond by C₁₋₄-alkyl or C₁₋₄-alkoxy or R₉ is in each case 1-naphthyl.

14. Ruthenium complexes of the formula VIII,
[(X)₂Ru(PR₁R₂R₃)(P-Z-N)] (VIII),
where
X is hydride, halide, C₁₋₈-alkoxide or C₁₋₈-acyloxy,
P-Z-N is a compound of the formula VI according to claim 12 or according to claim 13, and
R₁, R₂ and R₃ are each, independently of one another, a hydrocarbon radical or a C-bonded heterohydrocarbon radical having heteroatoms selected from the group consisting of O, S, N, NH and N(C₁-C₄-alkyl), each of which has from 1 to 22 carbon atoms, or R₁ and R₂ together with the phosphorus atom and further carbon atoms form a 4- to 8-membered ring and R₃ is as defined above, with R₁, R₂ and R₃ being unsubstituted or substituted.

15. Use of the ruthenium complexes of the formula VIII according to claim 14 for the hydrogenation of aldehydes and preferably prochiral ketones by means of hydrogen.
